(19) United States
(12) Patent Application Publication
(10) Pub. No.: US 2017/0224977 A1
(43) Pub. Date: Aug. 10, 2017

(54) URINARY CATHETER IRRIGATION DEVICE AND METHOD OF IRRIGATING A URINARY CATHETER

(71) Applicant: Webb Medical LLC, Philadelphia, PA (US)

(72) Inventor: Daniel Hyun Shin, Los Angeles, CA (US)

(21) Appl. No.: 15/426,169

(22) Filed: Feb. 7, 2017

Related U.S. Application Data
(60) Provisional application No. 62/292,410, filed on Feb. 8, 2016.

(51) Int. Cl. A61M 39/22; A61M 3/02; A61M 39/10; A61M 25/00; A61F 5/442

(52) U.S. Cl. CPC A61M 39/223; A61M 25/0017; A61F 5/442; A61M 39/10; A61M 3/0254; A61M 2039/0202

(57) ABSTRACT

Disclosed are a device and a method for irrigating a urinary catheter connected to a drainage bag. The device is configured to be selectively operated in any one of a drainage state, an irrigation state, and a disposal state without requiring its disconnection from the drainage bag. The device includes a port for receipt of a syringe holding an irrigation fluid. When in the drainage state the device enables fluid from the patient to passively drain into the bag. When in the irrigation state the device isolates the catheter from the bag and enables the irrigation liquid to be introduced from the syringe into the catheter and the patient's bladder to produce a contaminated fluid and also enables the contaminated fluid to be withdrawn back into the syringe. In the disposal state the device isolates the catheter from the drainage bag and enables the contaminated fluid to be introduced into the discharge bag.

### CROSS-REFERENCE TO RELATED APPLICATIONS

This utility application claims the benefit under 35 U.S.C. §119(e) of Provisional Application Ser. No. 62/292,410 filed on Feb. 8, 2016, entitled Urinary Catheter Irrigation Device and Method of Irrigating a Urinary Catheter. The entire disclosure of this provisional application is incorporated by reference herein.

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISK

Not Applicable

### FIELD OF THE INVENTION

This invention relates generally to medical devices and more particularly to devices for irrigating a urinary catheter and for providing a method for irrigating a urinary catheter.

### BACKGROUND OF THE INVENTION

Urinary catheters are inserted for bladder drainage in more than five million patients a year in the United States. Irrigating those catheters is necessary in many situations. For example, irrigation is commonly performed to ensure that the catheter is not clogged, or to flush out blood or debris from the bladder, or to check that the catheter is in the correct location. The current method used by health care workers to irrigate a urinary catheter involves disconnecting the drainage bag from the catheter to flush the catheter with a syringe of irrigation fluid, such as water or saline. In particular, a taper-tip piston syringe, which is filled with irrigation fluid, is fitted to the proximal end of the catheter after it has been disconnected from the drainage bag to which it had been connected. The irrigation liquid is pumped into and out of the bladder or catheter by operation of the syringe's piston to thereby clear the bladder or catheter of any obstructing debris. Unfortunately, this process is technically difficult to perform, has a high change of spillage or splashing of contaminated fluids, and cannot easily be done in a sterile manner. Moreover, when the drainage bag is removed from the catheter, this “breaks” the closed-system of urinary drainage, increasing the chances of a catheter-associated infection.

The maintenance of a “closed-system” is part of the CDC guidelines on the prevention of catheter-associated urinary tract infections. As is known, healthcare associated infections account for nearly $45 billion in direct hospital costs, and catheter associated urinary tract infections (CAUTI) are the most common type of healthcare associated infections. Furthermore, Medicare has stopped reimbursing hospitals since 2008 for the additional hospital costs associated with CAUTI.

Thus, a need exists for apparatus and method which overcomes those disadvantage of the prior art. The subject invention does that by providing a device and method for irrigating a urinary catheter to which a drainage bag is connected and without requiring the disconnection of the catheter from the drainage bag, while permitting the irrigation to be performed in a fashion that maximizes ease and efficiency. Moreover, the subject invention further improves on the traditional catheter irrigation process by enabling the contaminated irrigation fluid (hereinafter the “contaminated fluid”) to be disposed of directly into the catheter drainage bag. This reduces the chance that contaminated fluid is spilled or splashed onto surrounding surfaces or onto the healthcare worker, thereby minimizing the chance of contamination of the healthcare worker or of the surrounding environment.

### SUMMARY OF THE INVENTION

In accordance with one aspect of this invention there is provided a device for irrigating a urinary catheter having a distal end extending into the bladder of a patient and a proximal end located outside of the patient. The device is configured to be interconnected between the proximal end of the catheter and a drainage receptacle. The device comprises a main passageway and an access port coupled to the main passageway. The access port is configured for receipt of a portion, e.g., the tip of a component, e.g., a syringe, containing an irrigation liquid. The device is configured to be selectively operated in any one of a drainage state, an irrigation state, and a disposal state without disconnection of the device from the urinary catheter or the drainage receptacle. The device, when in the drainage state, enables fluid from the bladder and the catheter to flow through the main passageway into the drainage receptacle without the fluid exiting through the access port. The device, when in the irrigation state, isolates the catheter from the drainage receptacle and enables the irrigation liquid to be introduced from the component through the access port into the catheter to produce a contaminated fluid and enables the contaminated fluid to be withdrawn through the access port into the component. The device, when in the disposal state, isolates the catheter from the drainage receptacle and enables the contaminated fluid to be introduced from the component through the access port into the drainage receptacle while preventing the contaminated fluid from gaining ingress into the bladder.

In accordance with one exemplary embodiment of this invention, the device additionally comprises a shunt passageway. The shunt passageway is configured when the device is in the disposal state to enable the contaminated fluid to be introduced from the component through the access port into the shunt passageway to flow into the drainage receptacle. For example, that exemplary embodiment additionally comprises an openable discharge port and a blocking member. The main passageway has a first portion in fluid communication with the catheter and a second portion in fluid communication with the drainage receptacle. The blocking member is configured when in an open state to enable fluid to directly flow through the main passageway from the first portion to the second portion and when in a closed state blocking the passageway to prevent the direct flow of fluid from the first portion to the second portion. The shunt passageway has an inlet end connected to the first portion of the main passageway upstream of the blocking member and an outlet end connected to the second portion of the main passageway downstream of the blocking member. The discharge port is located at the inlet end of the shunt passageway and is normally closed to isolate the shunt passageway from the first portion of the main passageway. The discharge port is configured to receive the portion of a component that has the discharge fluid within it to enable the discharge fluid to be introduced into the shunt passageway to flow through the shunt passageway into the second portion of the main passageway and into the drainage receptacle.

In accordance with another exemplary embodiment of this invention, the device comprises a three-way valve. The three-way valve includes a chamber and a movable member. The chamber has a first port in fluid communication with the catheter and a second port in fluid communication with the drainage receptacle. The access port is in communication with the chamber. The movable member is located within the chamber and configured when the device is in the drainage state to leave the first and second ports unblocked, whereupon fluid can flow from the catheter through the first port into the chamber and from there into the second port and then into the drainage receptacle. The movable member is configured when the device is in the irrigation state to leave the first port unblocked but blocking the second port, whereupon irrigation fluid introduced from the component through the access port can flow into the chamber and the first port from whence it can flow into the catheter to produce the contaminated fluid and enabling the contaminated fluid to be withdrawn through the access port into the component. The movable member is configured when the device is in the disposal state to block the first port leaving the second port unblocked, whereupon contaminated fluid introduced from the component through the access port can flow into the chamber and second first port from whence it can flow into the drainage receptacle.

In accordance with another aspect of this invention chamber of the three way valve is circular chamber, and wherein the movable member is a rotatable member having a circular periphery including a first, a second, and a third port. The first, second and third ports are in fluid communication with one another within the interior of the rotatable member. The first and third ports are spaced from each other on the periphery of the rotatable member, with the second port being located on the periphery between the first and third ports. The movable member is located within the chamber and is configured to be rotated to a position within the chamber wherein the device is in the drainage state with the first port of the rotatable member in fluid communication with the first port of the chamber and the third port of the rotatable member in fluid communication with the second port of the chamber. The moveable member is configured to be rotated to a position within the chamber wherein the device is in the irrigation state with the first port of the rotatable member in fluid communication with the access port and the second port of the rotatable member in fluid communication with the first port of the chamber. The moveable member is configured to be rotated to a position within the chamber wherein the device is in the disposal state with the third port of the rotatable member in fluid communication with the access port and the second port of the rotatable member in fluid communication with the second port of the chamber.

In accordance with another aspect of this invention there is provided a method for irrigating a urinary catheter having a distal end extending into the bladder of a patient and a proximal end located outside of the patient. The method basically entails providing a drainage receptacle with an irrigation device connected between the proximal end of the catheter and the drainage receptacle. The irrigation device comprises an openable access port. The device is operated in a drainage state, whereupon it enables fluid from the bladder to flow through the catheter and through it into the drainage receptacle without the fluid exiting through the access port. The device is operated in an irrigation state to isolate the catheter from the drainage receptacle, whereupon a portion of a component containing an irrigation liquid is introduced through the access port and the component is caused to introduce the irrigation liquid into the device to produce a contaminated fluid and thereafter the component is caused to draw the contaminated fluid into the component. The device is operated in a disposal state to isolate the catheter from the drainage receptacle, whereupon the component containing the contaminated fluid is introduced through the access port and the component caused to introduce the contaminated fluid through the device into the drainage receptacle while preventing the contaminated fluid from gaining ingress into the bladder.

In accordance with one preferred aspect of the method of this invention, the component is not removed from the access port between the operation of the device in the irrigation state and the operation of the device in the discharge state.

### BRIEF DESCRIPTION OF THE DRAWING FIGURES

FIG. 1 is an isometric view of one exemplary device for irrigating a urinary catheter constructed in accordance with this invention being shown connected to a conventional urinary catheter and to a conventional drainage receptacle, e.g., a bag, to form a closed system and also showing a conventional irrigation syringe which is used with the device to irrigate the catheter;

FIG. 2 is an enlarged longitudinal sectional view of the exemplary device shown in FIG. 1, wherein the device is set to its “drainage” mode wherein it enables the drainage of fluid from a patient's urinary bladder through the catheter and the device into the drainage bag;

FIG. 3 is a view similar to FIG. 2 but showing the device set to its “irrigation” mode wherein irrigation fluid is introduced into the device from the irrigation syringe to irrigate the catheter and the patient's bladder;

FIG. 4 is a view similar to FIG. 3 also showing the device in its “irrigation” mode wherein the contaminated fluid is withdrawn back into the syringe;

FIG. 5 is a view similar to FIGS. 3 and 4, but showing the device in its “disposal” mode wherein the contaminated fluid that had been withdrawn back into the syringe is introduced through the device into the drainage bag;

FIG. 6 is an isometric view similar to FIG. 1, but showing an alternative exemplary device, e.g., a three-way flap-valve based device, for irrigating a urinary catheter constructed in accordance with this invention, wherein the device is set to its “drainage” mode to enable fluid from a patient's urinary bladder to flow through the catheter into the device and from there into the drainage bag;

FIG. 7A is an enlarged sectional view taken along line 7A-7A of FIG. 6;

FIG. 7B is a sectional view taken along line 7B-7B of FIG. 7A;

FIG. 8A is a sectional view similar to FIG. 7A but showing the device of FIG. 6 set to its “irrigation” mode, wherein the device enables irrigation fluid to introduced into it from an irrigation syringe to irrigate the catheter and the patient's bladder;

FIG. 8B is a sectional view taken along line 8B-8B of FIG. 8A;

FIG. 9A is a sectional view similar to FIG. 8A but showing the device of FIG. 6 also set to its “irrigation” mode wherein the contaminated fluid is withdrawn back into the irrigation syringe;

FIG. 9B is a sectional view taken along line 9B-9B of FIG. 9A;

FIG. 10A is a view similar to FIG. 7A, but showing the device of FIG. 6 in its “disposal” mode, wherein the device enables the contaminated fluid which had been withdrawn back into the irrigation syringe to be introduced through the device into the drainage bag;

FIG. 10B is a sectional view taken along line 10B-10B of FIG. 10A;

FIG. 11 is an isometric view similar to FIG. 6, but showing still another alternative exemplary device, e.g., a three-way rotary-valve based device, for irrigating a urinary catheter constructed in accordance with this invention, wherein the device is set to its “drainage” mode to enable fluid from a patient's urinary bladder to flow through the catheter into the device and from there into the drainage bag;

FIG. 12A is an enlarged sectional view taken along line 12A-12A of FIG. 11;

FIG. 12B is a sectional view, similar to FIG. 12A, but showing the device of FIG. 11 set to its “irrigation” mode, wherein the device enables irrigation fluid to introduced into it from an irrigation syringe to irrigate the catheter and the patient's bladder;

FIG. 12C is a sectional view, similar to FIG. 12A also showing the device of FIG. 11 in its “irrigation” mode wherein the contaminated fluid is withdrawn back into the irrigation syringe; and

FIG. 12D is a sectional view, similar to FIG. 12A, but showing the device of FIG. 11 in its “disposal” mode, wherein the device enables the contaminated fluid to be introduced through the device into the drainage bag.

### DETAILED DESCRIPTION OF THE PREFERRED EMBODIMENTS

Referring now to the drawings wherein like characters refer to like parts, there is shown in FIG. 1 an exemplary embodiment of a device 20 for irrigating a urinary catheter 10 while the catheter is positioned within the bladder of a patient and connected to a drainage bag 12, and without requiring the disconnection of the drainage bag from the catheter. As will be described in detail later the device 20 is configured to be selectively operated in three modes or states. One mode is the “drainage” mode or state, wherein the device enables fluid from the patient's bladder to flow through catheter 10 into the device 20 and from there into the drainage bag 12. Another mode is the “irrigation” mode or state, wherein the device enables an irrigation fluid, e.g., water or saline, to be introduced into it from a component, e.g., a syringe, 14 to irrigate the catheter and the patient's bladder to clear the bladder or catheter of any obstructing debris and then to withdraw the resultant fluid (i.e., the “contaminated” fluid) back into the syringe, all without requiring the disconnection of the drainage bag from the device. Another mode is the “discharge” or “disposal” mode or state, wherein the device enables the contaminated fluid to be introduced into it from the syringe and from there directly into the drainage bag, while preventing the contaminated fluid from gaining ingress into the catheter or bladder.

Before describing the details of the device 20 a brief description of the catheter 10, the drainage bag 12 and the irrigating syringe 14 are in order. Thus, as can be seen in FIG. 1, the catheter 10 is a conventional urinary catheter having a distal end 10A which is configured to be disposed in the bladder of a patient (not shown). The proximal end of the catheter includes a drainage connector 10B. That connector 10B is configured to be coupled to the inlet tube 12A of a conventional drainage bag 12. However, in this case the connector 10B is coupled to the inlet of the device 20. The drainage bag is formed of a flexible material defining a hollow interior or chamber that is in fluid communication with the inlet opening 12A so that fluid, e.g., urine, blood, etc. from the patient can flow (drain) therein for collection.

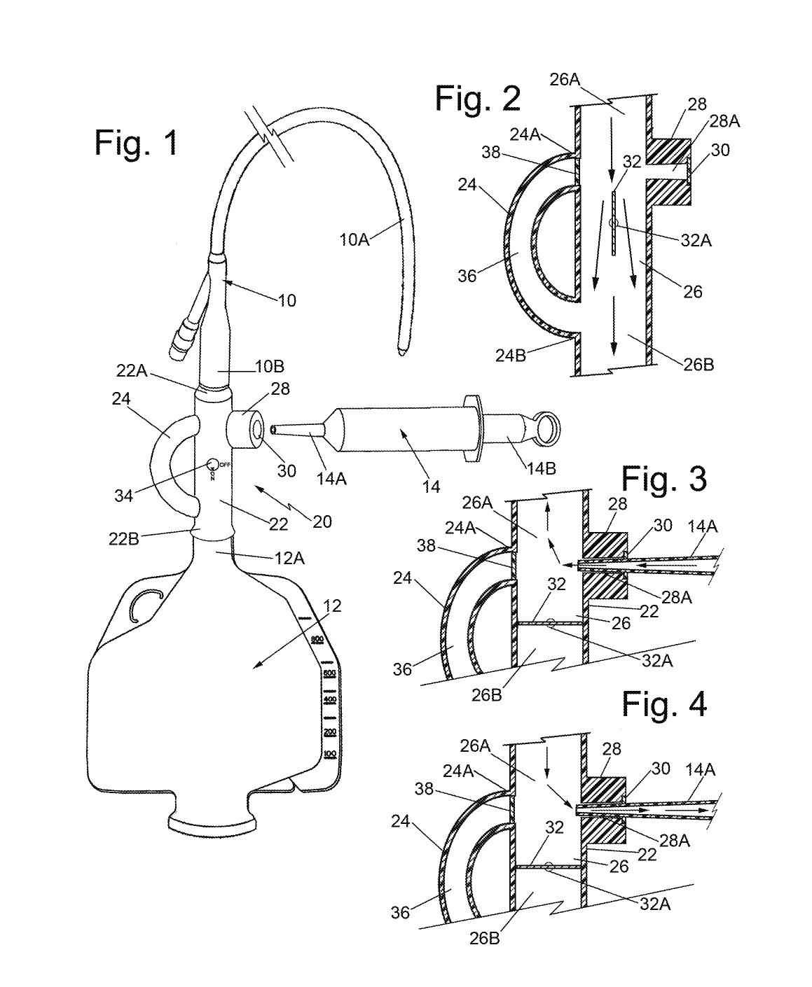

The device 20 is a somewhat elongated member, formed of any suitable material, e.g., plastic, having a main section 22 and a shunt section 24. The main section 22 has an inlet end 22A and an outlet end 22B. A main passageway 26 (FIG. 2) extends fully through the main section of the device from the inlet end 22A to the outlet end 22B. The inlet end 22A has a tapered tip and is configured to be inserted into the connector 10B at the proximal end of the urinary catheter 10 to form a good fluid-tight interface therebetween. To that end, the tapered tip may be crafted from an elastomeric material, e.g., silicone or rubber, for a secure, snug and fluid-tight fit of the tapered tip within the drainage connector 10B. Thus, fluid from the catheter can flow into the inlet end of the device and then into the main passageway 26 without leakage. The outlet end 22B is located at bottom of the device 20 and is slightly flared so that it can receive the inlet tube 12A of the drainage bag 12. To that end, the flared outlet end of the main section may also be crafted of an elastomeric material, e.g., silicone or rubber, for a secure, snug and fluid-tight fit of the drainage bag's inlet tube 12A within the drainage outlet end of the device. Thus, fluid from the passageway 26 can flow into the drainage bag without leakage at the interface.

The main section 22 of the device 20 includes a short projection 28 extending outward from one side of the main section 22. The projection 28 is a tubular member including an elongated access port 28A (FIG. 2) which serves as an irrigation port for the device. The access port is configured to receive the tip 14A of the irrigation syringe. As best seen in FIG. 2 the access port 28A includes a membrane 30 or any other similar structure closing off the end of the access port at the free end of the projection 28. The membrane is impermeable to the passage of liquid in its resting state. The opposite end of the access port 28A is located at the interface with the main passageway 26. The membrane 30 is normally closed, but is openable so that the tip 14A of the irrigation syringe 14 can be introduced through it into the access port, whereupon the tip of the syringe is in fluid communication with the main passageway 26 as shown in FIG. 3 and without fluid leaking out the interface between the membrane and the periphery of the tip.

As best seen in FIGS. 2-5 a movable blocking member, e.g., a pivotable disk 32 of circular profile, is located within the main passageway 26 just below the access port 28A. The disk is mounted on the end of a shaft 32A. The shaft 32A extends perpendicularly to the central longitudinal axis of the main passageway and through the wall of the main section so that the free end of the shaft is located outside the main section 22. The axis of the shaft 32A forms the pivot axis for the disk so that the disk can pivot about that axis. The location of the disk 32 within the main passageway defines the boundary or interface between an upper or upstream portion 26A of the main passageway and a lower or downstream portion 26B of the main passageway. A rotary knob 34 (FIG. 1) is secured to the free end of the shaft 32A and is configured to be rotated about the pivot axis to pivot the disk 32 between the open or “on” position or state shown in FIG. 2 and the closed or “off” position or state shown in FIGS. 3-5. When the knob 34 is rotated to the “off” position the peripheral edge of the disk makes tight contact with the inner surface of the main passageway 26 to isolate the upstream portion 26A of the main passageway from the downstream portion 26B. To that end, the outside diameter of the disk 32 is the same as the inside diameter of the passageway 26 and the disk is constructed so that its peripheral edge, when in engagement with the inner surface of the main passageway forms a good fluid-tight seal or interface therebetween. Accordingly, when the disk is pivoted to the “off” position, fluid is prevented from flowing from the upstream portion 26A of the central passageway to the downstream portion 26B. Conversely, when the disk is pivoted to the “on” position, fluid is enabled to flow from the upstream portion 26A of the central passageway to the downstream portion 26B.

The shunt section 24 basically comprises an arcuate tube having an upper end 24A fixedly secured to the main section 22 on the opposite side of the device as the projection 28 and which is axially aligned with the access port 28A. The shunt section includes a lower end 24B fixedly secured to the main section below the location of the disk 32. The shunt section is a hollow member defining a shunt passageway 36 extending fully through it. The lower end of the shunt passageway is in fluid communication with the downstream portion 26B of the main passageway 26. A normally closed, but openable discharge membrane 38, similar to the membrane 30, is located at the interface of the upper end of the shunt passageway 38 with the upstream portion 26A of the main passageway, whereupon the membranes 30 and 38 are axially aligned. The membrane 38, like the membrane 30, is impermeable to the passage of liquid in its resting state.

Use of the device 20 is as follows. The device 20 is connected in between the urinary catheter 10 and the drainage bag 12 at the time of the catheter's placement in the bladder of the patient. This can be done in a sterile fashion. In order to enable passive drainage of fluid from the patient's bladder all that is required is for the healthcare worker to rotate the knob 34 of the device 20 the “on” position, whereupon the device will be in its “drainage” mode or state. In particular, the rotation of the knob to the on position pivots the coupled disk 32 to the position shown in FIG. 2, whereupon fluid, e.g., urine, blood, etc., can flow from the patient's bladder, through the catheter into the upstream portion 26A of the main passageway as shown by the arrows in that figure, and from whence it can flow around the open disk 32 to the downstream portion 26B and hence into the drainage bag 12 for collection.

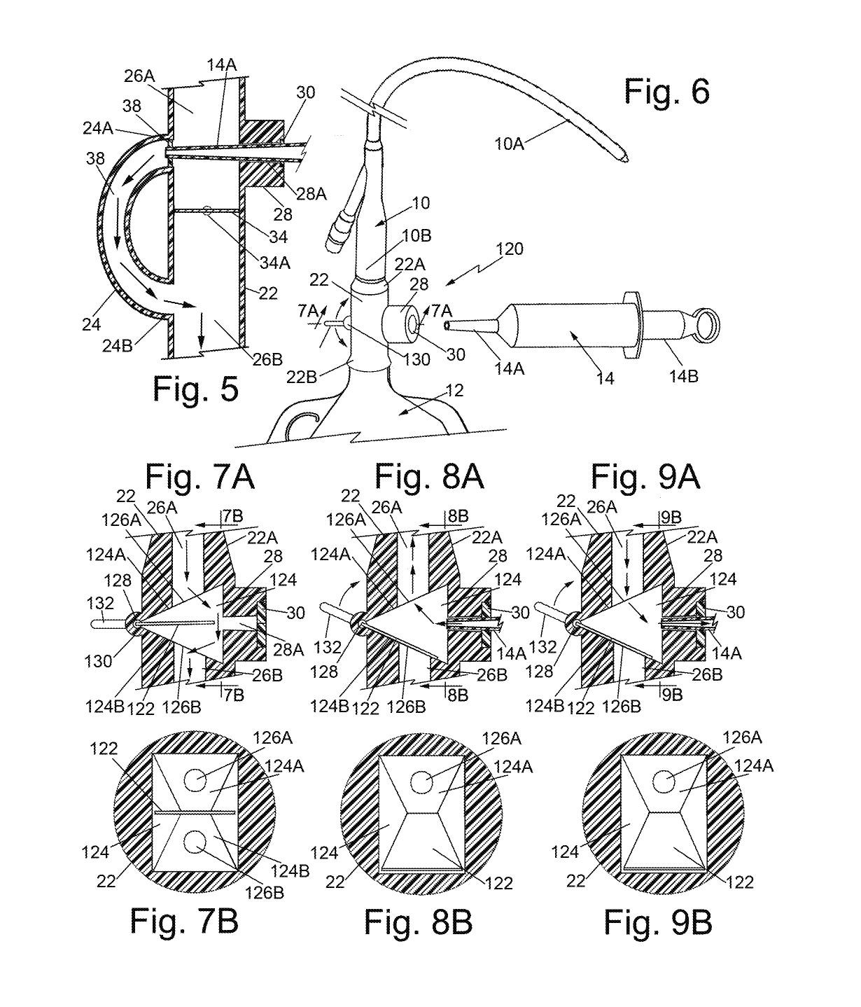

When bladder irrigation is desired, the healthcare worker prepares for that procedure by pouring a sterile irrigation fluid, e.g., saline or water, into a sterile basin, readies a sterile irrigation syringe 14, an alcohol prep pad, and dons sterile gloves. The alcohol prep pad is used to wipe the membrane 30 at the irrigation port 28A of the device. Then the syringe 14 is filled with the irrigation fluid by inserting its tip 14A into the irrigation fluid and pulling outward on the plunger 14B to draw the fluid into the interior of the syringe. The tip 14A of the syringe is then inserted through the membrane 30 into the irrigation port 28A so that the free end of the syringe's tip is in fluid communication with the upstream portion 26A of the central passageway, while the healthcare worker's other hand stabilizes the urinary catheter 10. The device 20 is then switched from its normal, “drainage” mode to the “irrigation” mode. In particular, all that the health care worker has to do is to rotate the knob 34 to the “off” position to thereby pivot the coupled disk 32 to the position shown in FIG. 3. This action isolates the upstream portion 26A of the main passageway from the downstream portion 26B and halts the drainage of fluid from the patient bladder into the drainage bag. The plunger 14B of the irrigation syringe can then be depressed by the healthcare worker to force the irrigation liquid out of the syringe and into the upstream portion 26A of the main passageway. Inasmuch as the discharge membrane 38 is normally closed, and the disk 32 is in its closed state isolating the upstream portion 26A of the main passageway from the downstream portion 26B, the irrigation fluid injected by the syringe into the upstream portion 26A will be forced upward into the catheter to irrigate it and the patient's bladder. That action produces the heretofore defined contaminated fluid, e.g., a mixture of urine, debris, the irrigation fluid, etc. The plunger 14B of the syringe can then be pulled back by the healthcare worker. This action draws the contaminated fluid from the bladder and catheter and through the upstream portion of the main passageway, back into the syringe via its tip 14A such as shown by the arrows in FIG. 4. Once that has been accomplished, and without removing the tip of the irrigation syringe from the access port membrane 30, the tip 14A of the irrigation syringe can be forced deeper into the device 20 by the healthcare worker so that it passes through the discharge membrane 38 into the upper portion of the shunt passageway 36 as shown in FIG. 5, whereupon the device will be in its discharge or disposal mode or state. Then the plunger 14B of the syringe can be pressed to force the discharge fluid into the shunt passageway, from whence it will flow into the downstream portion 26B of the main passageway and into the drainage bag 12.

If necessary, the irrigation process can be repeated. For example, after the contaminated fluid is introduced into the drainage bag through the device 20, the irrigation syringe can be removed from the access port 28A and the syringe refilled with the irrigation fluid. After that has been accomplished with the knob 34 still in the “off” position, the tip 14A of the refilled irrigation syringe can be reinserted through the membrane 30 into the access port 28A like shown in FIG. 3, and the irrigation process as described above repeated.

Once the catheter and the patient's bladder have been irrigated sufficiently to clear any debris, the knob 34 can be rotated back to the “on” position so that the device is back in the drainage mode. This action permits passive drainage of urine from the patient's bladder through the catheter and the device into the drainage bag as described above.

Turning now to FIG. 6, there is shown an alternative embodiment of a device 120 for irrigating a urinary catheter 10 constructed in accordance with this invention. The device 120 is also configured to be selectively operated in the three modes or states as described with reference to device 20 and is constructed somewhat similarly to that device. In the interest of brevity the features of the device 120 which are common to the device 20 will be given the same reference numbers and their structural and operational details will not be reiterated. The major difference between the device 120 and the device 20 is that the device 120 does not include a shunt passageway and the movable blocking member, e.g., pivotable disk 34. Instead, as can be clearly seen in FIG. 7A, the device 120 includes a flap valve 122 located within a generally pyramidal shaped cavity 124 in the main section 22 immediately adjacent the access port 28A and between the upstream portion 26A of the main passageway 26 and the downstream portion 26B. That flap valve 122 is a thin, planar member formed of a somewhat flexible material, e.g., silicone, rubber, etc. The cavity 124 includes an upwardly sloping planar top wall 124A and a downwardly sloping planar bottom wall 124B. The top wall 124A has an opening or port 126A in it which is in fluid communication with the main passageway portion 26A. The bottom wall 124B has an opening or port 126B in it which is in fluid communication with the main passageway portion 26B. The flap 122 is fixedly secured to one end of a shaft 128. The shaft 128 is laterally offset from the central longitudinal axis of the main passageway 26 and extends perpendicularly to that axis of the main passageway and through another projection 130 in the main section 22. The projection 130 is located opposite the projection 28. A pivotable lever or handle 132 is secured to the free end of the shaft 128. The axis of the shaft 128 forms the pivot axis for the flap 122 so that the flap can pivot about that axis upon rotation of the lever or handle 132 in either the clockwise or counter-clockwise direction. When the handle is in the middle position like shown in FIGS. 1, 7A and 7B, the device will be in its normal, “drainage” state or mode, wherein the flap 122 will be oriented so that it is directed towards the membrane 30 leaving the ports 126A and 126B uncovered and hence open. Thus, fluid from the patient's bladder can flow through the catheter into the upstream portion 26A of the main passageway, through the open port 126A into the cavity 124, around the flap 122, and out through the open port 126B into the downstream portions 26B of the main passageway from whence it can flow into the drainage bag 12.

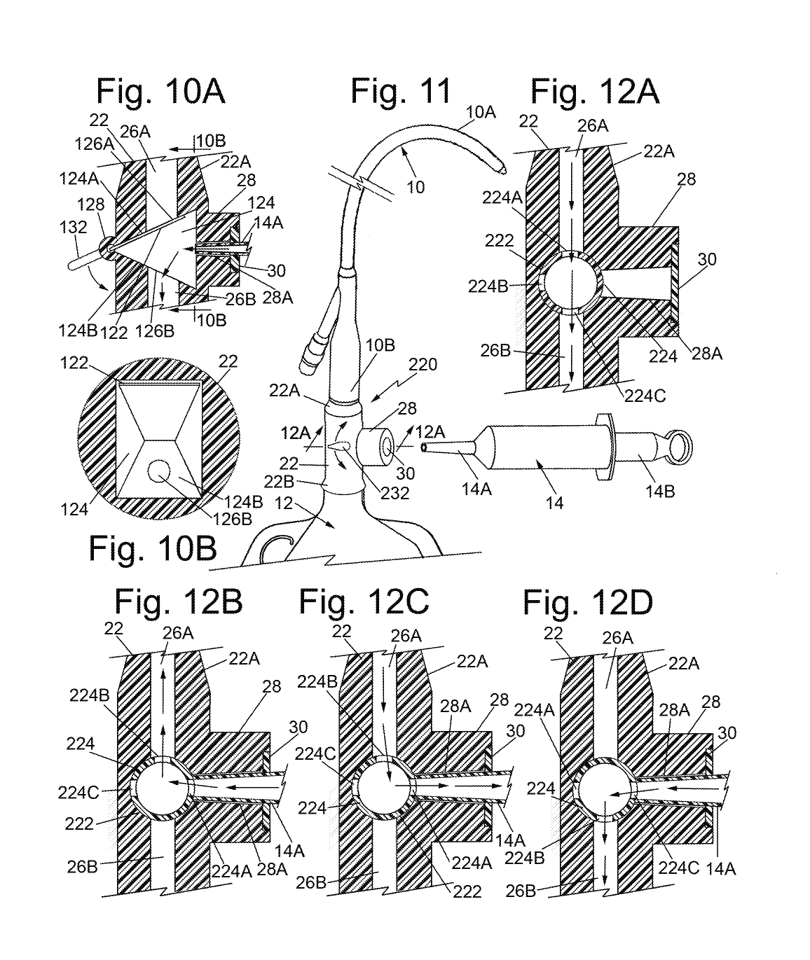

Use of the device 120 to irrigate the catheter and the patient's bladder is as follows. The access port membrane 30 wiped down with the alcohol prep pad and the irrigation syringe is prepared and filled as described above. The lever 132 is pivoted to the up position shown in FIGS. 8A and 8B, whereupon the flap 122 is pivoted downward so that it makes contact with the bottom wall 124B of the cavity 124 contiguous with the port 126B, thereby closing off or sealing that port. This action isolates the upstream portion 26A of the main passageway from the downstream portion 26B and halts the drainage of fluid from the patient bladder into the drainage bag. The tip 14A of the syringe is then inserted by the healthcare worker through the membrane 30 into the irrigation port 28A so that the free end of the syringe's tip is in fluid communication with the interior of the cavity 124. The plunger 14B of the irrigation syringe can then be depressed by the healthcare worker to force the irrigation liquid out of the syringe and into the cavity 124. Inasmuch as the port 126B is now closed by the engagement of the flap 122 with the wall 124B surrounding the port 126B, the irrigation fluid injected by the syringe into the cavity will be forced upward through the open port 124A into the upstream portion 26A of the main passageway as shown by the arrows in FIG. 8A and hence into the catheter to irrigate it and the patient's bladder. That action produces the heretofore defined contaminated fluid. The plunger 14B of the syringe can then be pulled back by the healthcare worker to draw the contaminated fluid from the bladder and catheter and through the upstream portion of the main passageway, back into the syringe via its tip 14A such as shown by the arrows in FIG. 9A. Once that has been accomplished, and without removing the tip of the irrigation syringe from the access port membrane 30, the lever 132 can be pivoted to the down position shown in FIG. 10A, whereupon the device will be in its “discharge” or “disposal” mode or state. In that state the flap 122 is pivoted upward so that it makes contact with the top wall 124A of the cavity 124 contiguous with the port 126A, thereby closing off or sealing that port, while opening the port 126B. Once that has been accomplished the plunger 14B of the syringe can be pressed to force the contaminated fluid into the cavity 124, from whence it will flow through the open port 126B into the downstream portion 24B of the main passageway and into the drainage bag 12. Moreover, the closure of the port 126A by the flap 122, when in the “discharge” state will prevent any of the contaminated fluid injected into the cavity 124 from gaining ingress back into the catheter.

If necessary, the irrigation process can be repeated using the device 120. For example, after the discharge fluid is introduced into the drainage bag through the device 120, the irrigation syringe can be removed from the access port 28A and the syringe refilled with the irrigation fluid. After that has been accomplished with the lever 132 still in the down position, like shown in FIG. 10A the tip 14A of the refilled irrigation syringe can be reinserted through the membrane 30 into the access port 28A like shown in FIG. 8A and the irrigation process as described above repeated.

Once the catheter and the patient's bladder have been irrigated sufficiently to clear any debris, the lever 132 can be rotated back to the intermediate position shown in FIGS. 6 and 7A so that the device is back in the drainage mode. This action permits passive drainage of urine from the patient's bladder through the catheter and the device into the drainage bag as described above.

Turning now to FIG. 11, there is shown still another alternative embodiment of a device 220 for irrigating a urinary catheter 10 constructed in accordance with this invention. The device 220 is also configured to be selectively operated in the three modes or states as described with reference to device 120 and is constructed somewhat similarly to that device. In the interest of brevity the features of the device 220 which are common to the devices 20 and 120 will be given the same reference numbers and their structural and operational details will not be reiterated. The major difference between the device 220 and the device 120 is that the device 220 does not include the flap valve. Instead, the device 220 includes three position rotary valve. In particular, the main section 22 of the device 220 includes a circular cavity 222 located immediately adjacent the access port 28A and between the upstream portion 26A of the main passageway 26 and the downstream portion 26B. The circular cavity is in fluid communication with the access port 28A. Located within the circular cavity 222 is a rotatable member 224, e.g. a hollow circular shaped tube section. That tube section is sealed at each end and incudes three openings or ports 224A, 224B and 224C in the sidewall of the tube. The ports 224A and 224C are disposed about the periphery of the tube section so that they lie diametrically opposite each other, with the port 224B being disposed between the ports 224A and 224B at a ninety degree angle to each. Thus, the ports are all in fluid communication with one another within the interior of the tube section. It should be noted that the rotatable member 224 need not be a tubular member, e.g., it can be a solid member so long as the ports 224A, 224B and 224C are in fluid communication with one another inside the member 224. The rotatable member 224 is configured to be rotated within the cavity 222 about a pivot axis which extends perpendicularly to central longitudinal axis the main passageway 26 and which is centered in the cavity. One end of the rotatable member 224 is connected to a pivotable handle or lever 232 (FIG. 11) which is located on the outside of the main section 22 of the device 220. The lever 232 is configured to be pivoted in the clockwise direction from the neutral position shown in FIG. 11 wherein the device is in its normal, or “drainage” state, to an upward position wherein the device is in its “irrigation” state or to a downward position wherein the devices is in its “discharge” or “disposal” state.

The device 220 is connected in between the urinary catheter 10 and the drainage bag 12 at the time of the catheter's placement in the bladder of the patient like that done in the usage of the devices 20 and 120. To enable passive drainage of fluid from the patient's bladder all that is required is for the healthcare worker to rotate the lever 232 of the neutral position, like shown in FIG. 11, whereupon the device will be in its “drainage” mode or state. In particular, the rotation of the lever to the neutral position rotates the circular rotatable member 224 to the position shown in FIG. 12A, whereupon fluid, e.g., urine, blood, etc., can flow from the patient's bladder, through the catheter into the upstream portion 26A of the main passageway as shown by the arrows in that figure, and from whence it can flow through the port 224A into the rotatable member 224, out through the port 224C into the downstream portion 26B and hence into the drainage bag 12 for collection.

When irrigation of the catheter is desired, all that the healthcare worker has to do is to wipe down the access port membrane 30 with the alcohol prep pad and prepare and fill the irrigation syringe as described above. When that has been accomplished the lever 232 can be pivoted upward to set the device in its “irrigation” state. In this state the port 224A of the tube section 224 will be in fluid communication with the access port 28A, the port 224B will be in fluid communication with the upstream portion 26A of the main passageway and a peripheral portion of the tube section will be blocking the downstream portion 26B of the main passageway. This action isolates the upstream portion 26A of the main passageway from the downstream portion 26B and halts the drainage of fluid from the patient bladder into the drainage bag. The tip 14A of the syringe can then be inserted by the healthcare worker through the membrane 30 into the irrigation port 28A so that the free end of the syringe's tip is in fluid communication with the port 224A. The plunger 14B of the irrigation syringe can then be depressed by the healthcare worker to force the irrigation liquid out of the syringe and into the access port 28A, whereupon that fluid can flow in the direction of the arrows shown in FIG. 12B, through the port 224A, into the tube section 224 and out through the port 224B, from whence it can flow through the upstream portion 26A of the main passageway into the catheter and the patient's bladder to irrigate the catheter and the patient's bladder to thereby produce the contaminated fluid. The plunger 14B of the syringe can then be pulled back by the healthcare worker. This action draws the contaminated fluid from the bladder and catheter and through the upstream portion of the main passageway, back into the syringe via its tip 14A such as shown by the arrows in FIG. 12C. Once that has been accomplished, and without removing the tip of the irrigation syringe from the access port membrane 30, the lever 232 can be pivoted to the down position, whereupon the device will be in its “discharge” or “disposal” mode or state. In that state the port 224C of the tube section 224 will be in fluid communication with the access port 28A, the port 224B will be in fluid communication with the downstream portion 26B of the main passageway and a peripheral portion of the tube section will be blocking the upstream portion 26B of the main passageway. Once that has been accomplished the plunger 14B of the syringe can be pressed to force the contaminated fluid into the access port 28A, whereupon that fluid can flow in the direction of the arrows shown in FIG. 12D, through the port 224C, into the tube section 224 and out through the port 224B, from whence it can flow through the downstream portion 26B of the main passageway into the drainage bag 12. Moreover, the closure of the upstream portion 26A by the engagement of a peripheral portion of the tube section 224 with it when the device is in the “discharge” or “disposal” state will prevent any of the contaminated fluid injected into the cavity device from gaining ingress back into the catheter.

If necessary, the irrigation process can be repeated using the device 220. For example, after the discharge fluid is introduced into the drainage bag through the device 220, the irrigation syringe can be removed from the access port 28A and the syringe refilled with the irrigation fluid. After that has been accomplished with the lever 232 still in the up position so that the device is still in its “discharge” or “disposal” state, the tip 14A of the refilled irrigation syringe can be reinserted through the membrane 30 into the access port 28A like shown in FIG. 12B and the irrigation process as described above repeated.

Once the catheter and the patient's bladder have been irrigated sufficiently to clear any debris, the lever 232 can be rotated back to the intermediate position shown in FIG. 11 so that the device 220 is back in the “drainage” mode. This action permits passive drainage of urine from the patient's bladder through the catheter and the device into the drainage bag as described above.

It should be pointed out at this juncture that the exemplary embodiments shown and described above are merely a few of various modifications to the device that can be made in accordance with this invention. What is important is that the device include some openable and closeable access port and some means for enabling the device to be selectively operated in the drainage, irrigation and disposal modes while a drainage receptacle is connected to the device, and wherein the device includes an access port configured to accept some component to introduce an irrigation liquid into the device during the irrigation mode to produce the contaminated fluid and to withdraw the contaminated fluid from the device so that it can be reintroduced into the device during the disposal mode. For example, a device constructed in accordance with this invention may be configured for use with a component, other than a syringe, to introduce an irrigation liquid into the device while enabling the “contaminated” fluid, to be withdrawn into the component for subsequent introduction back into the device and to the drainage receptacle connected to the device.

As should be appreciated by those skilled in the art from the foregoing, the device and method of the subject invention allow for bladder irrigation via urinary catheters to be performed in a fashion that maximizes ease and efficiency, while minimizing chance of contamination of the healthcare worker or the surrounding environment. Moreover, the device of the subject invention enables one to establish a “closed” catheter drainage system including a catheter and a collection receptacle, wherein the system can remain intact, without disconnection of any portion, during normal drainage operation as well as during irrigation and disposal operation. Thus, device and method of the subject invention allows for faster and more hygienic irrigation of urinary catheters, providing benefits for healthcare workers, patients, and hospitals.

Without further elaboration the foregoing will so fully illustrate my invention that others may, by applying current or future knowledge, adopt the same for use under various conditions of service.

I claim:

1. A device for irrigating a urinary catheter having a proximal end extending into the bladder of a patient and a distal end located outside of the patient, said device being configured to be interconnected between the proximal end of the catheter and a drainage receptacle, said device comprising a main passageway and an access port coupled to said main passageway, said access port being configured for receipt of a portion of a component containing an irrigation liquid, said device being configured to be selectively operated in any one of a drainage state, an irrigation state, and a disposal state without disconnection of said device from the urinary catheter or the drainage receptacle, said device when in said drainage state enabling fluid from the bladder and the catheter to flow through said main passageway into the drainage receptacle without said fluid exiting through said access port, said device when in said irrigation state isolating the catheter from the drainage receptacle and enabling the irrigation liquid to be introduced from the component through said access port into the catheter to produce a contaminated fluid and enabling the contaminated fluid to be withdrawn through said access port into the component, said device when in said disposal state isolating the catheter from the drainage receptacle and enabling the contaminated fluid to be introduced from the component through said access port into the drainage receptacle while preventing the contaminated discharge fluid from gaining ingress into the bladder.

2. The device of claim 1 wherein said device additionally a shunt passageway, said shunt passageway being configured when said device is in said disposal state to enable the contaminated fluid to be introduced from said component through said access port into said shunt passageway to flow into the drainage receptacle.

3. The device of claim 2 wherein said device additionally comprises an openable discharge port and a blocking member, said main passageway having a first portion in fluid communication with the catheter and a second portion in fluid communication with the drainage receptacle, said blocking member being configured when in an open state to enable fluid to directly flow through said main passageway from said first portion to said second portion and when in a closed state blocking said passageway to prevent the direct flow of fluid from said first portion to said second portion, said shunt passageway having an inlet end connected to said first portion of said main passageway upstream of said blocking member and an outlet end connected to said second portion of said main passageway downstream of said blocking member, said discharge port being located at said inlet end of said shunt passageway and being normally closed to isolate said shunt passageway from said first portion of said main passageway, said discharge port being configured to receive a portion of a component having the discharge fluid within the component to enable the discharge fluid to be introduced into said shunt passageway to flow through said shunt passageway into said second portion of said main passageway and into said drainage receptacle.

4. The device of claim 3 wherein said access port is located at one side of said first portion of said main passageway and wherein said discharge port being located on an opposite side of said first portion of said main passageway and aligned with said access port, whereupon said portion of the component can be inserted through said access port and through said discharge port.

5. The device of claim 1 wherein said device comprises a portion of urinary catheter system, and wherein said urinary catheter system includes the catheter and the drainage receptacle.

6. The urinary catheter system of claim 5 wherein said drainage receptacle comprises a flexible bag.

7. The device of claim 1 wherein said device comprises a three-way valve.

8. The device of claim 7 wherein said three way valve includes a chamber and a movable member, said chamber having a first port in fluid communication with the catheter and a second port in fluid communication with the drainage receptacle, said access port being in communication with said chamber, said movable member being located within said chamber and configured when said device is in said drainage state to leave said first and second ports unblocked, whereupon fluid can flow from the catheter through said first port into said chamber and from there into said second port and then into the drainage receptacle, said movable member being configured when said device is in said irrigation state to leave said first port unblocked but blocking said second port, whereupon irrigation fluid introduced from a portion of the component through the access port can flow into said chamber and said first port from whence it can flow into the catheter to produce the contaminated fluid and enabling the contaminated fluid to be withdrawn through said access port into the component, said movable member being configured when said device is in said discharge state to block said first port leaving said second port unblocked, whereupon the contaminated fluid introduced from the component through the access port can flow into said chamber and second first port from whence it can flow into the drainage receptacle.

9. The device of claim 8 wherein said movable member comprises a flap and wherein said chamber includes a first valve seat surrounding said first port and a second valve seat surrounding said second port, said flap being configured to engage said second valve seat when said device is in said irrigation state and to engage said second valve seat when said device is in said discharge state, said flap being configured to be spaced from and out of engagement with said first and second valve seats when said device is in said drainage state.

10. The device of claim 9 wherein said flap is planar and wherein each of said first and second valve seats is planar.

11. The device of claim 7 wherein said device comprises a portion of urinary catheter system, and wherein said urinary catheter system includes the catheter and the drainage receptacle.

12. The device of claim 8 wherein said chamber is circular chamber, and wherein said movable member is a rotatable member having a circular periphery including a first, a second and a third port, said first, second and third ports being in fluid communication with one another within the interior of said rotatable member, said first and third ports being spaced from each other on said periphery of said rotatable member, with said second port being located on said periphery between said first and third ports, said movable member being located within said chamber and configured to be rotated to a position within said chamber wherein said device is in said drainage state with said first port of said rotatable member in fluid communication with said first port of said chamber and said third port of said rotatable member in fluid communication with said second port of said chamber, said moveable member being configured to be rotated to a position within said chamber wherein said device is in said irrigation state with said first port of said rotatable member in fluid communication with said access port and said second port of said rotatable member in fluid communication with said first port of said chamber, said moveable member being configured to be rotated to a position within said chamber wherein said device is in said disposal state with said third port of said rotatable member in fluid communication with said access port and said second port of said rotatable member in fluid communication with said second port of said chamber.

13. The device of claim 12 wherein said device comprises a portion of urinary catheter system, and wherein said urinary catheter system includes the catheter and the drainage receptacle.

14. A method for irrigating a urinary catheter having a proximal end located within the bladder of the patient and proximal end located outside of the patient, said method comprising:
a) providing a drainage receptacle;
b) connecting an irrigation device between said proximal end of said catheter and said receptacle, said device comprising an openable access port;
c) operating said device in a drainage state, whereupon said device enables fluid from the bladder to flow through said catheter and said device into said drainage receptacle without said fluid exiting through said access port,
d) operating said device in an irrigation state to isolate said catheter from said drainage receptacle;
e) introducing a portion of a component containing an irrigation liquid through said access port and causing said component to introduce said irrigation liquid into said device to produce a contaminated fluid and thereafter causing said component to draw said contaminated fluid into said component; and
f) operating said device in a disposal state to isolate said catheter from said drainage receptacle; to cause said component to introduce said discharge fluid through said device into said drainage receptacle while preventing said contaminated fluid from gaining ingress into the bladder.

15. The method of claim 13 wherein said component is not removed from said access port between the operation of said device in said irrigation state and the operation of said device in said discharge state.

16. The method of claim 14 wherein said component comprises a syringe.

17. The method of claim 16 wherein the method additionally comprises refilling said syringe with an irrigation fluid after said contaminated fluid had been introduced by said syringe into said drainage receptacle and repeating the operation of the device in the irrigation state and then the discharge state.
